(19) United States
(12) Patent Application Publication
(10) Pub. No.: US 2013/0165775 A1
(43) Pub. Date: Jun. 27, 2013

(54) MEDICAL INJECTION DEVICE AND METHOD FOR CONTROLLING THE MEDICAL INJECTION DEVICE

(71) Applicants: STEFAN ASSMANN, Erlangen (DE); BJÖRN HEISMANN, Erlangen (DE); RETO MERGES, Erlangen (DE); MARKUS SCHMIDT, Nuernberg (DE); SEBASTIAN SCHMIDT, Weisendorf (DE); KERA WESTPHAL, Berlin (DE)

(72) Inventors: STEFAN ASSMANN, Erlangen (DE); BJÖRN HEISMANN, Erlangen (DE); RETO MERGES, Erlangen (DE); MARKUS SCHMIDT, Nuernberg (DE); SEBASTIAN SCHMIDT, Weisendorf (DE); KERA WESTPHAL, Berlin (DE)

(21) Appl. No.: 13/716,299

(22) Filed: Dec. 17, 2012

(30) Foreign Application Priority Data
Dec. 22, 2011 (DE) 102011089620.1

(51) Int. Cl. G06Q 50/24; A61M 5/00

(52) U.S. Cl. CPC G06Q 50/24; A61M 5/007
USPC 600/432

(57) ABSTRACT

An injection device and methods for controlling the injection device are provided. Patient data for a patient to be treated are provided and an injection preparation (injectable) used in the injection device is automatically identified by the injection device automatically reading a label provided on a container of the injection preparation. Furthermore, contraindications for an identified injection preparation are determined. The patient data are compared with the any contraindication that has been determined At least one protective measure is adopted to prevent the injection when at least one contraindication matches the patient data. According to another variant of the method, patient data for a patient to be treated and contraindications for the injection preparations (injectables) available for a selection are established. The patient data are compared with the contraindications, wherein at least one injection preparation is suggested which contraindications do not match the patient data.

### CROSS REFERENCE TO RELATED APPLICATIONS

This application claims priority of German Patent Application No. 10 2011 089 620.1 DE filed Dec. 22, 2011. All of the applications are incorporated by reference herein in their entirety.

### FIELD OF INVENTION

An injection device and a method for controlling the injection device are provided.

### BACKGROUND OF INVENTION

Injection devices are used in particular in image-generating medical technology, for example in computer tomography (CT), rotation angiography, magnetic resonance tomography (MR), positron emission tomography (PET) or single-photon emission computed tomography (SPECT), in order to inject a contrast medium into the body of a patient who is to be examined, before or during image capture and in measured doses. Furthermore, injection devices are also used for slow discharge of drugs, that is, extending over a fairly long period, and for the automatically controlled discharge of infusions. The generic term “injection preparation” is used hereafter for the substance to be injected, that is in particular, the contrast medium, the drug or the infusion solution.

In a conventional design, such an injection device is embodied for the automatic emptying of a syringe or other kind of capsule containing the injection preparation. Such an injection device, which is also known as a “syringe pump”, usually includes a mounting into which the syringe or capsule filled with injection preparation can be placed. The injection device further includes an advancing mechanism, by means of which the syringe piston having an adjustable advance movement can be pushed in or the volume of injection preparation can be compressed in another way such that the injection preparation contained in the syringe or capsule can be discharged at a predeterminable rate.

In an alternative version, used in particular for the administration of infusions, the injection device includes a pump, by means of which the injection preparation can be discharged in a controlled manner from a reservoir container.

In both versions, the injection device additionally contains a control unit, by means of which the discharge rate of the injection preparation and/or the discharge period can be adjusted.

In image-generating medical technology in particular, the contrast media used here as injection preparations have to be selected extremely carefully, in order to achieve optimum effectiveness whilst avoiding side effects and complications as far as possible, for example, as a consequence of allergies. This selection is usually left to the medical staff. The contrast medium to be used is typically selected on the basis of a patient interview, relating in particular to the height and weight of the patient, and to existing intolerances. If this information is incomplete or incorrect, because, for example, the patient has not been asked about certain risk factors or withholds important information in ignorance of the interactions, this may lead to the selection of an unsuitable contrast medium, and as a result thereof to sometimes drastic complications. For example, in a patient suffering from renal insufficiency, the administration of contrast media containing gadolinium may cause serious disease progressions, for example, nephrogenic systemic fibrosis, with in some cases permanent disabilities or even fatal consequences.

A method and a device in which patient data on file are compared with the data for the injection preparation in order to reduce the level of complications, are known, for example, from DE 10 2005 046 784 A1.

### SUMMARY OF INVENTION

An object is to further develop an injection device and a method for the control thereof in such a way that the risk of injecting the wrong injection preparation is reduced. The object is achieved by methods and a device as claimed in the claims.

According to a first provided method, patient data relating to a patient who is to be treated are established from a hospital information system. Furthermore, according to the method, an injection preparation used in the injection device (that is, inserted into the injection device) is identified, that is, recognized by what type it is. For example, either directly or indirectly, a product identification explicitly assigned to the injection preparation and/or the type and amount or concentration of an active ingredient contained in the injection preparation is established. Finally, contraindications of the injection preparation that has been identified are established from a database of medicinal products, for example. The patient data are now automatically compared with the contraindication or with each contraindication that has been established. A protective measure that is directed at the prevention of the injection is adopted if the comparison described in the aforementioned results in at least one contraindication applying to the patient information.

According to a second provided method, patient data for a patient to be treated being are established from a hospital information system. Furthermore, contraindications for a range of injection preparations available for selection are established, for example, from a database of medicinal products. In this embodiment, the patient data are again compared with the contraindication or with each contraindication that has been established. Here, however, the result of this comparison is evaluated such that at least one of the (optionally a plurality of) injection preparations where the contraindications do not apply to the patient data is suggested for use in an injection. All injection preparations where the contraindications do not apply to the patient data are preferably suggested for use in an injection.

In the second variant of the method, in order to avoid overloading the corresponding list of suggestions, that is therefore, to minimize the number of injection preparations that are deemed suitable and thus allow a user to make a simple and targeted selection, the injection preparations available for selection have preferably been automatically pre-selected from a fairly wide range of basically available injection preparations according to predetermined treatment instructions, that is, according to instructions regarding the type of medical treatment or investigation intended. The treatment instructions may also consist in particular in the specifying of an image-generating medical modality or investigation method (for example, CT, PET, MR, SPECT, etc.) or include such a modality or method. On the basis of these instructions, only contrast media that may be used with this modality or investigation method are preselected and considered in the further procedure. For example, where the treatment instructions are for “PET”, only radiopharmaceuticals that emit the positron rays detected in PET (indirectly via the corresponding annihilation radiation) are selected as injection preparations.

Treatment instructions may be provided in particular automatically to the injection device by the modality assigned thereto. To this end, the injection device is preferably connected to the assigned modality by communications technology.

Both variants of the method which are described in the aforementioned are based on the shared knowledge that, by comparing the patient data obtained from the hospital information system with the contraindications, those injection preparations that are linked to a possible risk for the patient to be treated can be ruled out in a fail-safe manner.

Both variants of the method which are described in the aforementioned may be carried out in isolation from each other, that is, separately. Preferably, however, these variants are used in combination with each other. This involves, according to the second variant of the method, one or a plurality of injection preparations where the contraindications do not apply to the patient data initially being suggested for the injection. After successful insertion of an injection preparation into the injection device, the injection preparation used is then identified (as in the first variant of the method). In the process a check is carried out as to whether the injection preparation used matches the suggested injection preparation or (where a plurality of injection preparations have been suggested) one of the suggested injection preparations. If this is not the case, a protective measure is adopted to prevent the injection being administered (again as in the first variant of the method).

In both variants of the method, the identification of the injection preparation used in the injection device is achieved automatically, by the injection device automatically reading a label affixed to a container of the injection preparation. This label is in particular a bar code or color code, what is known as an RFID tag or such like, which clearly identifies the type of injection preparation used.

As a protective measure adopted to prevent the injection being administered, provision can be made where an incorrect (unsuitable) injection preparation has been identified, for the injection device to generate and emit an acoustic, visual or other form of warning that is detectable by a user of the device. This warning preferably consists of the issuing of an acoustic warning sound in combination with a verbal message that is issued on a visual display unit assigned to the injection device and that alerts the user to the lack of suitability of the injection preparation that is being used.

In a particularly fail-safe embodiment of the method, as an alternative to or in addition to the warning, provision can be made for the injection device, as a protective measure when an unsuitable injection preparation is identified, to automatically block the discharge (injection) thereof. This block can be achieved for example, by the advancing drive or the pump in the injection device being automatically deactivated.

In a development of the variants of the method, an injection rate and/or an injection interval are/is established by comparing the patient data relating to the patient to be treated with a set of instructions for the use of an injection preparation that has been identified or suggested. Corresponding sets of instructions for use are preferably stored in a database of medicinal products. In the preferred variant of the method the injection device is automatically adjusted to the injection rate and/or to the injection interval that have been established. As an alternative thereto, provision can indeed also be made for the injection rate or the injection interval that have been established to be issued to the user as a suggestion for the manual adjustment of the injection device.

In a further embodiment of the method, provision is made using a set of instructions (stored in a database of medicinal products, for example, as described in the aforementioned) for use of the injection preparation that has been identified or suggested, for a desired temperature of the injection preparation to be established automatically. In this case provision is preferably made for a temperature control unit in the injection device to be automatically set to the desired temperature that has been established. Alternatively, it is also possible for the desired temperature that has been established to be issued to the user as a suggestion for the corresponding adjustment of the injection device.

The injection device includes a control unit that is equipped with circuitry technology and/or program technology enabling it to carry out the method automatically in one of the variants of the method described in the aforementioned. The control unit may consist of a microcontroller that is incorporated in the injection device, in which controller the instructions required to carry out the method automatically are provided in the form of control software and are ready to run.

In an alternative embodiment, the control unit is purely a software module that can also be used independently of the injection device, for example, on a control computer pertaining to the image-generating modality.

To carry out the method, the injection device, or at least the control unit thereof is preferably connected to the hospital information system by communications technology in order to establish the patient data. To establish the contraindications, and likewise optionally the set of instructions for use, the injection device, or at least the control unit thereof, is preferably connected to a database of medicinal products. Furthermore, the injection device is preferably connected by communications technology to the image-generating modality.

### BRIEF DESCRIPTION OF THE DRAWINGS

FIG. 1 shows an image-generating medical modality in a schematic block diagram, shown here in the form of an X-ray C-arm system having an injection device assigned thereto and likewise a hospital information system and a database of medicinal products, which are connected with the C-arm system and the injection device via a communications network.

FIG. 2 shows a schematic block diagram showing in greater detail the injection device according to FIG. 1.

FIGS. 3 to 5 show three variants of a method for the control of the injection device.

The same reference signs are used consistently in all the figures to denote parts and values that are equivalent to one another.

### DETAILED DESCRIPTION OF INVENTION

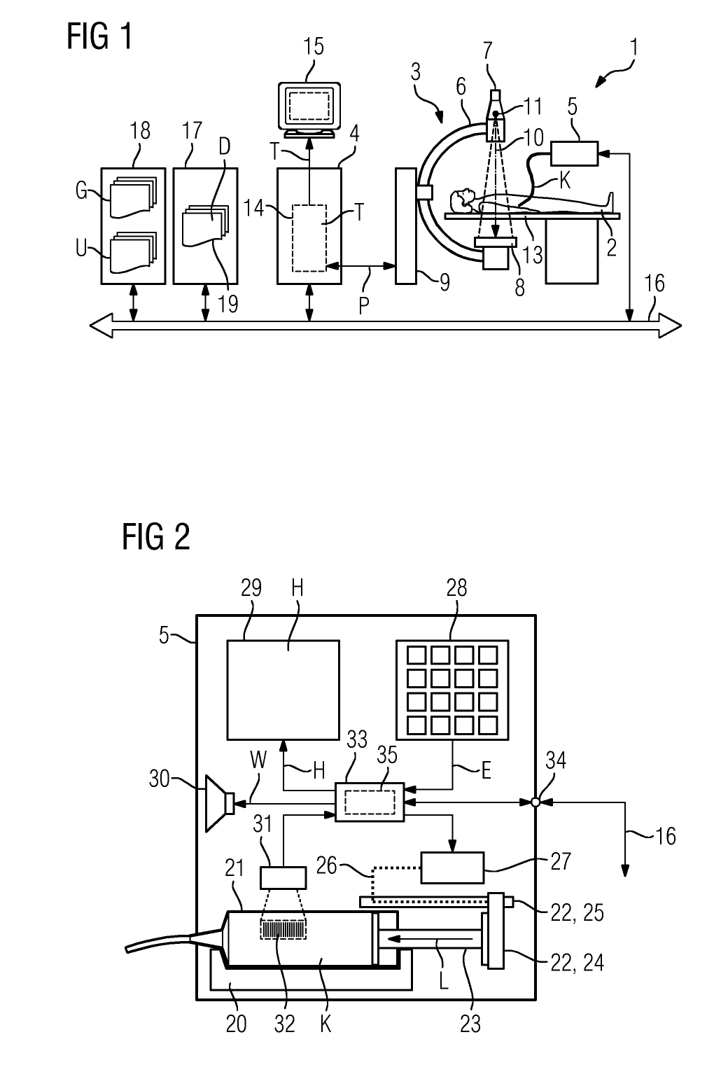

FIG. 1 shows a device 1 for contrast-assisted capturing of image data from inside the body of the patient 2. The device 1 includes as an image-generating modality an (X-ray) C-arm device 3, for example in rotation angiography, and likewise a control and evaluation data processor 4. The device 1 further includes an injection device 5 assigned to the C-arm device 3.

The C-arm device 3 includes a C-arm 6, at both ends of which an X-ray source 7 or an X-ray detector 8 are mounted facing each other. The C-arm 6 is in turn supported on a base 9. The C-arm is rotatable round a horizontal axis with respect to said base 9. It is additionally pivotable along the line of the arc—that is, in the plane defined by the C-arm.

A central beam 10 of the C-arm device 3 can thus be adjusted in virtually any orientations with respect to the surrounding space. Here the central beam 10 denotes the spatial vector which connects a focusing mechanism 11 of the X-ray source 7 to a center of the X-ray detector 8, and which in particular is aligned vertically to the surface of the detector. The central beam 10 forms the center of a cone-shaped X-ray beam, which center is projected onto the two-dimensional detector surface of the X-ray detector 8 during the operation of the C-arm device 3, starting out from the focusing mechanism 11.

Instead of the C-arm device 3 described in the aforementioned, the device 1 can, however, also include a different image-generating modality, which can be used in contrast-assisted image generation, for example, a computer tomograph (CT), a magnetic resonance (MR) tomograph, a positron-emission tomograph (PET), or a single photon emission computed tomograph, (SPECT for short). Merely for the sake of simplification and without limiting the generality, reference will be made hereafter exclusively to the C-arm device 3.

Assigned to said device is a patient couch 13 on which the patient 2 can be placed in such a way that the patient's body region to be investigated is arranged in the image capture zone of the C-arm device 3.

During the operation of the device 1, the C-arm 6 is moved round the body region to be captured in the patient 2 who is placed on the patient couch 13, a plurality of projected images of this body region being captured from various projection directions using the X-ray source 7 and the X-ray detector 8. The C-arm device 3 submits a projection image data set P comprising these projection images to the computer 4.

In the data processor 4, software 14 is implemented, which is used firstly to activate the C-arm device 3, and secondly to prepare the projection image data set P, the software 14 being configured in particular for the reconstruction (back projection) of the projection image data set P into a three-dimensional image data set of the inside of the body of the patient 2. The three-dimensional image data set will be referred to hereafter as a tomogram T.

The completed tomogram T can be stored locally in the data processor 4 or in an external image archive (not shown explicitly). Additionally or alternatively, displayable, two-dimensional representations can be generated from the tomogram T, for example, layered image representations or visualized (“generated”) volume representations, and displayed on a visual display unit 15.

The C-arm device 3 and/or the control and evaluation data processor thereof 4 and the injection device 5 are incorporated into a communications network 16, in particular into what is known as a Local Area Network (LAN) and via this network 16 are able to exchange data bilaterally with further data processing components of a medical installation. In particular, the C-arm device 3 or the data processor thereof 4 and the injection device 5 are linked up via the network 16 with a Hospital Information System 17 (HIS for short) and also with a database of medicinal products 18. In the Hospital Information System 17, electronic patient files 19 are managed with patient data D. The database of medicinal products 18 stores in particular contraindications G and sets of instructions for use U in order to dose out and use the medicinal products available. The information stored in the database of medicinal products 18 includes, in particular, contraindications G and sets of instructions for use U relating to the contrast media K used by the injection device 5 as injection preparations.

The design of the injection device 5, which, in the example shown is configured in the style of an injection pump, is illustrated in greater detail in FIG. 2. According to this representation, the injection device 5 contains a mounting 20, into which a syringe 21 filled with the contrast medium K that is to be injected can be placed. The injection device 5 further includes an advancing mechanism 22, by means of which a piston 23 in the syringe 21 can be pushed forward to empty said syringe. In the embodiment used by way of example, the advancing mechanism 22 includes a slider 24, which is linearly guided along a track 25 in the longitudinal direction L of the syringe 21 that is housed in the mounting 20. The slider 24 is coupled here by means of an adjustment mechanism 26 (merely indicated in FIG. 2) to an electric motor 27 and can be pushed forward towards the syringe 21 at an adjustable speed with the aid of the motor 27.

In the embodiment according to FIG. 2, the injection device 5 additionally includes a keypad 28 with which a user of the device 1 can make alphanumeric inputs E, a display panel (hereafter referred to as display 29) to issue alphanumeric suggestions H and also to confirm the user's inputs E, and likewise a speaker 30, through which acoustic warnings W (in particular in the form of warning tones) can be issued to the user. The injection device 5 additionally contains a reading device 31 (in the form of a barcode-reader here), with which a label applied onto the syringe 21 (in particular a barcode 32) can be read electronically.

The injection device 5 also includes a control unit 33 to control the electric motor 27, the keypad 28, the display 29, the speaker 30 and the reading device 31. The control unit 33 is additionally connected via a network interface 34 to the communications network 16.

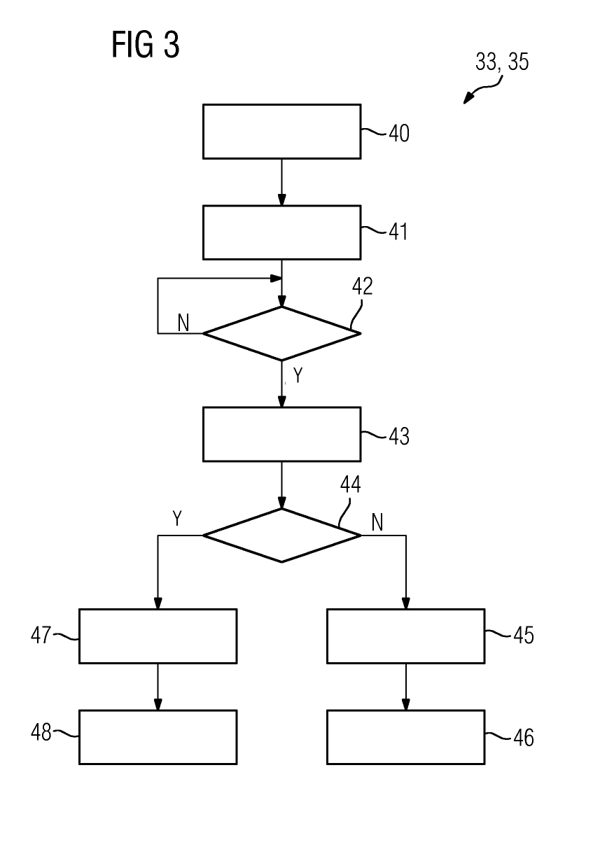

In the preferred embodiment, the control unit 33 is essentially made up of a microcontroller in which control software 35 is incorporated in a ready to run format. This control software 35 and hence the control unit 33 enable the method sketched out in diagram form in FIG. 3 to be carried out automatically when the injection device 5 is operated according to specifications. This method is automatically carried out prior to each injection of a contrast medium K into the body of the patient 2 administered using the injection device 5. The injection can therefore only be administered after successful completion of the method.

In the preferred embodiment, the method according to FIG. 3 is carried out automatically by the data processor 4 of the C-arm device 3 in the run-up to a contrast-assisted image capture, the name of the patient 2 to be examined or alternatively a patient identification number being supplied via the network 16 to the injection device 5 in a (method) step 40 by the data processor 4. As an alternative thereto, it is also possible for a user of the device 1 to initiate the method by inputting the name of the patient 2 or the patient's patient identification number into the injection device 5 via the keypad 28.

In a (method) step 41, using the patient's name or patient identification number, the control software 35 then loads, via the network 16, the patient data D for the patient 2 from the patient file 19 stored in the Hospital information system 17.

In a further (method) step 42, the control software 35 checks whether a labeled syringe 21 has been placed into the mounting 20 of the injection device 5 and whether a bar code 32 can be read accordingly by the reading device 31. The syringe 21 is preferably a ready-made capsule of contrast medium, which has already been pre-filled by the manufacturer with a specific contrast medium K. In this case, the bar code 32 that clearly identifies the contrast medium K has usefully been securely affixed already by the manufacturer onto the syringe 21, such that, when the syringe 21 has been placed into the mounting 20 according to specifications, the code is located in the field of view of the reading device 31.

Alternatively, it is also possible for the syringe 21 to be a standard disposable syringe, which is not specified for a particular contrast medium K, and into which is inserted the contrast medium K that is to be used for the injection in each case. In this case the bar code 32 is usefully applied onto an adhesive label which is stuck onto the syringe 21 by the user of the device 1 after a specific contrast medium K has been inserted.

Step 42 is repeated by the control software 35 until no more contrast medium K can be identified using the reading device 31 (N).

Conversely, as soon as a specific contrast medium K has been identified (Y), in a (method) step 43, the control software 35 loads the contraindications G stored for this contrast medium K with the aid of the name of the contrast medium K that has been identified from the database of medicinal products 18.

In a subsequent (method) step 44, the control software 35 checks whether any of the contraindications G that have been uploaded for the contrast medium K that has been identified match the patient data D uploaded for the patient 2. If, for example, the contraindications G that have been uploaded contain the instruction that the contrast medium K should not be used where the patient 2 suffers from renal insufficiency, the control software 35 checks against the patient data D as to whether the patient 2 suffers from renal insufficiency. If on the other hand the contraindications that have been uploaded G specify that the contrast medium K should not be used with patients who are below a certain age, the control software 35 then uses the date of birth contained in the patient data D to check whether the patient 2 is of a sufficient age for the contrast medium K to be administered.

Insofar as this check produces a negative result (N), that is, insofar as none of the contraindications G that have been uploaded match the patient data D, the control software releases the injection in a (method) step 45. In this case, in a (method) step 46, the control software 35 loads from the database of medicinal products 18 the set of instructions for use U that have been stored for the contrast medium K that has been identified and sets the advancing speed of the motor 27 and the temperature of a temperature control unit that is incorporated in the mounting 20 (not shown in more detail) according to the advice given in the set of instructions for use U that have been uploaded.

Otherwise (Y), that is, insofar as one of the contraindications G that have been uploaded matches the uploaded patient data D, the control software 35 cuts off the motor 27, in a (method) step 47, such that the motor 27 cannot be set in motion by different control instructions given by the user or the data processor 4.

In this case, in order to make the user aware of this cut-off, the control software issues the acoustic warning W via the speaker 30, in a (method) step 48. Furthermore, the control software 35 issues the advice H via the display 29. The advice H is, for example, a text message which indicates that the contrast medium K that has been identified must not be used on the patient 2 because of the contraindication G.

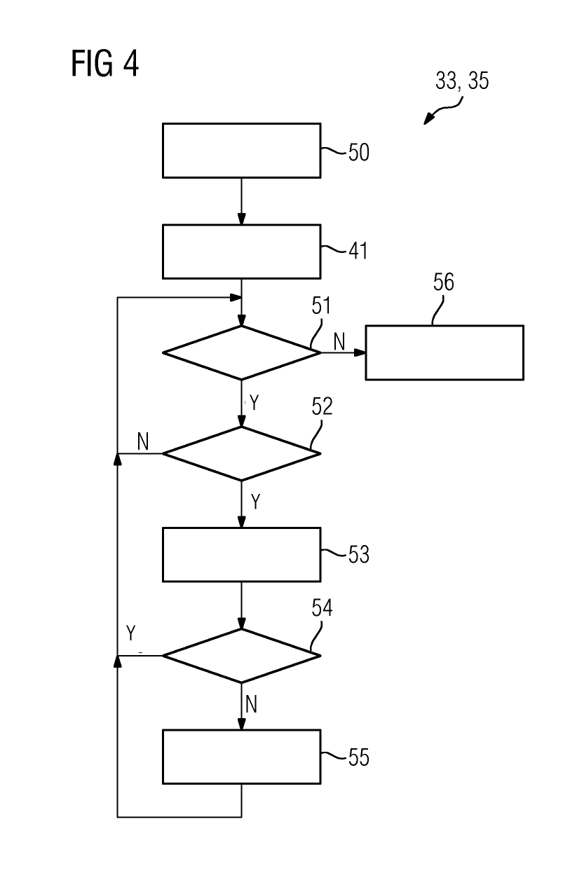

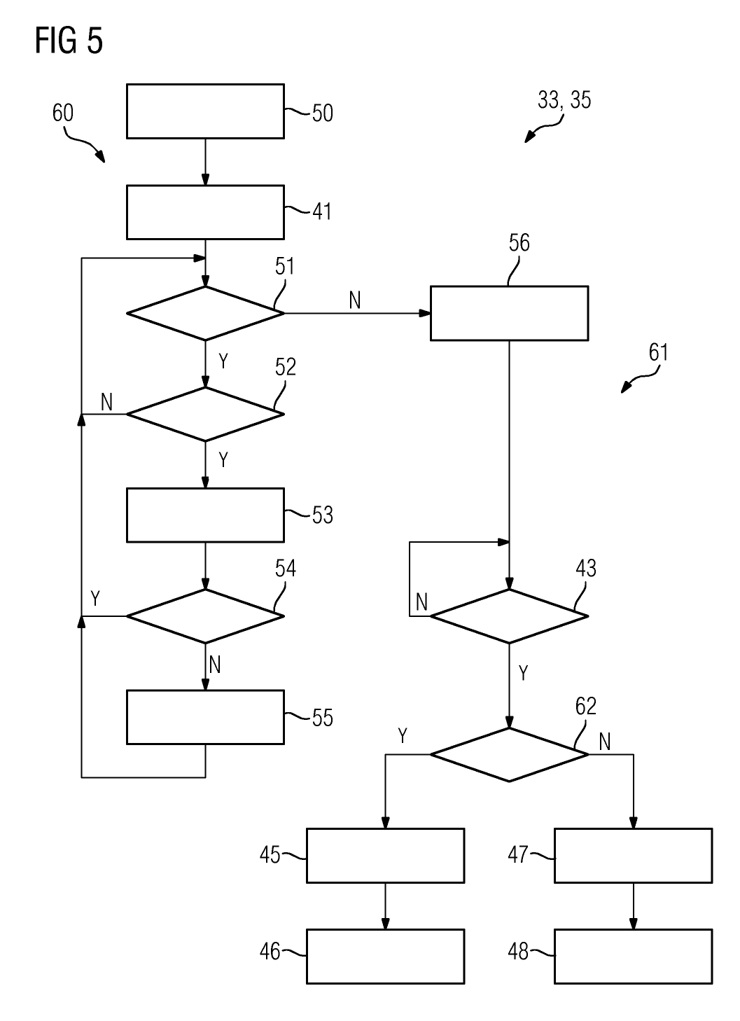

An alternative variant of the method is shown in FIG. 4. This method differs from the variant of the method described in the aforementioned in that the control software 35 does not react to the selection of a certain contrast medium K by the user, but the control software 35 is already activated before this selection is made and supports the user therein. In this variant, too, the method is again initiated by the data processor 4 in a (method) step 50, the name of the patient 2 or a corresponding patient identification number being transmitted to the control software 35 via the network 16.

Additionally, in a useful embodiment of the method, a “treatment instruction”, specifying the type of modality and likewise optionally containing in addition an instruction about the type of investigation planned using this modality, is transmitted to the control software 35. In the example shown, the treatment instruction that the control software 35 receives consists of the information that the image-generating modality is the C-arm device 3.

Subsequent to step 50, the control software 35 reads the patient data D for the patient 2 from the Hospital Information System 17 with the method step 41 already described in connection with FIG. 3. Next, the control software 35 checks in a subsequent (method) step 51 whether a set of instructions for use U of a contrast medium K can be read from the database of medicinal products 18. This condition is regularly met the first time method step 51 is carried out.

Optionally (Y), by comparing the set of instructions for use U that has been read with the treatment instruction, the control software 35 checks in a subsequent (method) step 52 whether the corresponding contrast medium K is usable in the modality that is assigned to the injection device 5, here that is, to the C-arm device 3 and (if this information is provided) whether the planned type of treatment can be carried out. If this is not the case (N), the control software 35 reverts to step 51.

When method step 51 is repeated, this involves the contrast media K listed in the database of medicinal products 18 being worked through in turn. The nth time that (n=1, 2, 3, and so on) step 51 is carried out, the control software 35 therefore attempts to read the set of instructions for use U of the nth contrast medium K listed in the database of medicinal products 18.

If the verification carried out in step 52 has a positive outcome (Y), that is, if according to the set of instructions for use U that has currently been read, the contrast medium K under examination is compatible with the modality specified in the treatment instruction and with the optionally specified type of investigation, then in a (method) step 53 arranged downstream, the control software 35 reads the contraindications G for the contrast medium K under consideration from the database of medicinal products 18.

In a subsequent (method) step 54, the control software 35 checks (as in method step 44 according to FIG. 3) whether any of the contraindications G that have been uploaded for the contrast medium K in question match the patient data D for the patient 2. Insofar as this is the case (Y), the control software 35 reverts to step 51 and consequently reads the set of instructions for use G for the next contrast medium K listed in the database of medicinal products 18 (insofar as this is available).

Otherwise, where none of the contraindications G that have been uploaded match the patient data D for the patient 2 (N), in a (method) step 55, the control software 35 stores a clearly identifiable name for the contrast medium K in question, storing in particular the product name of the contrast medium or a product number in a suggestion list. Next, the control software again reverts to step 51 and consequently reads the set of instructions for use U for the next contrast medium K listed in the database of medicinal products 18 (insofar as this is available). This software loop is run until all the contrast media K listed in the database of medicinal products 18 have been worked through, and consequently the check carried out in step 51 produces a negative result (N).

In this case, in a (method) step 56, the control software 35 issues the suggestion list generated in step 55, via the display 29, which list is filled successively with names denoting the suitable contrast media K after the software loop described in the aforementioned has been run a plurality of times. Alternatively provision can also be made for the injection device 5 to transmit the suggestion list to the data processor 4 via the network 16 for display on the visual display unit 15 or print it out using a printer (not shown in more detail).

With the aid of the suggestion list, the user can select in a fail-safe manner a contrast medium K that is suitable both for the investigation that is to be carried out using the C-arm device 3 and for the individual patient 2.

To make the method more fail-safe, in a third variant of the method that is shown in FIG. 5, the variants of the method described in the aforementioned with reference to FIG. 3 and FIG. 4 are combined with each other. Here, a first method step 60 of the method according to FIG. 5 is similar to the method shown in FIG. 4. The final step thereof, step 56, that is, the issuing of the suggestion list containing the contrast media K that have been found to be suitable is followed according to FIG. 5 by a second method step 61 that essentially corresponds to the method according to FIG. 3. In the context of this method step 61, the control software 35 starts by carrying out method step 42. The control software 35 therefore checks whether, after the issue of the suggestion list, the user of the device 1 has meanwhile selected a contrast medium K and whether this can be identified by the reading device 31. As in the method according to FIG. 3, step 42 is repeated until a contrast medium K has been identified.

Subsequently, in a (method) step 62, which replaces step 44 of the method according to FIG. 3, a check is carried out as to whether the contrast medium K identified in step 43 corresponds with a contrast medium K included in the suggestion list. Insofar as this is the case (Y), it is ensured that none of the contraindications G for the contrast medium K that has been identified corresponds with the patient data D for the patient 2. In this case, as in the method according to FIG. 3, the injection is released by the control software 35 (step 45) and the advancing speed of the electric motor 27 and also the desired temperature in the temperature control unit are set according to the set of instructions for use U for the contrast medium K that has been identified (step 46).

Otherwise, if that is, the contrast medium K identified in step 43 does not correspond with any of the contrast media K contained in the suggestion list (N), the control software 35 cuts off the electric motor 27 (step 47) and issues a corresponding warning W in addition to a corresponding alert (step 48).

The variants of the method described with the aid of FIGS. 3 to 5 are preferably carried out fully automatically in the control unit when the control software 35 is run, that is without active input by the user.

The control software 35 described in the aforementioned as a functional component of the control unit 33 can alternatively be used outside the injection device 5, in particular as part of the software 14 in the data processor 4.

The subject matter of the invention is not restricted to the exemplary embodiments described in the aforementioned. In fact, a person skilled in the art can derive further embodiments of the invention from the description given in the aforementioned. In particular, the individual features of the invention and the design variants thereof described with the aid of the various exemplary embodiments can also be used in different combinations with one another.

1. A method of controlling an injection device, comprising:
providing patient data for a patient to be treated,
automatically identifying an injection preparation for an injection device, wherein the injection device automatically reads a label applied onto a container of the injection preparation,
determining contraindications of the injection preparation which have been identified,
comparing the patient data with the contraindications which have been determined, and
adopting a protective measure in order to prevent an injection when at least one contraindication matches the patient data.

2. The method of controlling an injection device,
providing patient data for a patient to be treated,
identifying contraindications of available injection preparations,
comparing the patient data with the contraindications which have been identified, and
suggesting at least one injection preparation with contraindications which are inapplicable to the patient data.

3. The method as claimed in claim 2, wherein the available injection preparations are automatically preselected from a plurality of injection preparations in accordance with a predetermined treatment instruction.

4. The method as claimed in claim 2, further comprising:
inserting the at least one injection preparation into an injection device,
checking whether the at least one injection preparation that has been inserted into the injection device corresponds with a suggested injection preparation,
adopting at least one protective measure in order to prevent an injection when the at least one injection preparation that has been inserted into the injection device does not correspond with the suggested injection preparation.

5. The method as claimed in claim 4, wherein the at least one injection preparation used in the injection device is automatically identified.

6. The method as claimed in claim 1, wherein a warning signal is issued as the protective measure.

7. The method as claimed in claim 4, wherein a warning signal is issued as the protective measure.

8. The method as claimed in claim 1, wherein the injection of the injection preparation is automatically blocked as a protective measure.

9. The method as claimed in claim 4, wherein the injection of the injection of the injection preparation is automatically blocked as a protective measure.

10. The method as claimed in claim 1, wherein an injection rate and an injection interval are established by comparing the patient data with a predetermined set of instructions for use of the injection preparation that has been identified or suggested.

11. The method as claimed in claim 2, wherein an injection rate and an injection interval are established by comparing the patient data with a predetermined set of instructions for use of the injection preparation that has been identified or suggested.

12. The method as claimed in claim 10, wherein the injection device is automatically adjusted to the injection rate and the injection interval which have been established.

13. The method as claimed in claim 11, wherein the injection device is automatically adjusted to the injection rate and the injection interval which have been established.

14. The method as claimed in claim 1, further comprising:
determining a temperature of the injection preparation based upon a predetermined set of instructions for use of the injection preparation that has been identified or suggested.

15. The method as claimed in claim 2, further comprising:
determining a temperature of the injection preparation based upon a predetermined set of instructions for use of the injection preparation that has been identified or suggested.

16. The method as claimed in claim 14, wherein a temperature control unit in the injection device is automatically adjusted to the temperature which has been determined.

17. The method as claimed in claim 15, wherein a temperature control unit in the injection device is automatically adjusted to the temperature which has been determined.

18. An injection device, comprising:
a control unit which is configured to automatically carry out a method as claimed in claim 1.
